(19) United States
(12) Patent Application Publication
(10) Pub. No.: US 2024/0175096 A1
(43) Pub. Date: May 30, 2024

(54) Visual loop-mediated isothermal amplification (LAMP) method for the rapid test of tobacco

(71) Applicant: YUNNAN TOBACCO QUALITY SUPERVISION AND TESTING STATION, Kunming (CN)

(72) Inventors: Ke ZHANG, Kunming (CN); Dan CHEN, Kunming (CN); Haiyan LI, Kunming (CN); Zhijun TONG, Kunming (CN); Chunqiong WANG, Kunming (CN); Jinchu YANG, Kunming (CN); Shichun QIN, Kunming (CN); Haowei SUN, Kunming (CN); Xiaowei ZHANG, Kunming (CN); Jie LONG, Kunming (CN)

(73) Assignee: YUNNAN TOBACCO QUALITY SUPERVISION AND TESTING STATION, Kunming (CN)

(21) Appl. No.: 18/306,286

(22) Filed: Apr. 25, 2023

(30) Foreign Application Priority Data
Nov. 30, 2022 (CN) 202211528367.1

(51) Int. Cl. C12Q 1/6895

(52) U.S. Cl. CPC C12Q 1/6895

(57) ABSTRACT

A visual loop-mediated isothermal amplification (LAMP) method for a rapid test of tobacco includes: extracting a genomic DNA of the tobacco, designing primers, establishing a LAMP reaction system, and optimizing the LAMP reaction system. The present disclosure designs the LAMP primers according to a conserved region of a screened tobacco-specific Ntsp151 genomic sequence, and establishes the LAMP method based on color determination. The present disclosure can rapidly identify flue-cured tobacco leaves, finished cigarettes, and tea cigarettes, and achieves a detection limit of 200 copies per reaction. The test results of the LAMP method for non-tobacco samples are negative, indicating that the LAMP method has high specificity. Compared with the quantitative polymerase chain reaction (qPCR) method, the LAMP method has a coincidence rate of 96.7%, but the LAMP method only needs about 1 h to obtain the results.

### CROSS REFERENCE TO THE RELATED APPLICATIONS

This application is based upon and claims priority to Chinese patent application no. 202211528367.1, filed on Nov. 30, 2022, the entire contents of which are incorporated herein by reference.

### SEQUENCE LISTING

The instant application contains a sequence listing which has been submitted in XML format via EFS-Web and is hereby incorporated by reference in its entirety. Said XML copy is named GBDHHY010-PKG_Sequence_listing.xml, created on 04/07/2023, and is 7,323 bytes in size.

### TECHNICAL FIELD

The present disclosure belongs to the technical field of molecular biological identification and test methods, and in particular, relates to a visual loop-mediated isothermal amplification (LAMP) method for a rapid test of tobacco.

### BACKGROUND

As an important cash crop, tobacco is the main raw material for preparing commercial cigarettes. Under the conditions of the market economy, the tobacco industry has gradually evolved from simple rapid growth to high-quality development, and its importance has become increasingly apparent. Tobacco identification is an important challenge for tobacco inspection. Tobacco identification is mainly implemented by molecular biological methods, such as sequence-characterized amplified region (SCAR) markers, random amplification polymorphic DNA (RAPD), amplified fragment length polymorphism (AFLP), simple sequence repeat (SSR), and single nucleotide polymorphism (SNP). These techniques often require complex devices such as polymerase chain reaction (PCR) and genome sequencing devices, which to some extent limits their application in the inspection of front-line tobacco products in monopoly enforcement.

### SUMMARY

To solve the above problems, the present disclosure provides a visual LAMP method for a rapid test of tobacco.

The present disclosure is implemented by the following technical solution.

The visual LAMP method for a rapid test of tobacco includes: extracting a genomic DNA of the tobacco, designing primers, establishing a LAMP reaction system, and optimizing the LAMP reaction system.

Further, the extracting a genomic DNA of the tobacco includes: weighing and adding 10-15 mg of a tobacco sample into an Eppendorf (EP) tube, adding 5% of a Chelex-100 suspension, grinding with a grinding rod for 1-2 min, and shaking and suspending for 10-30 s; adding 1/10 volume of proteinase K and 1/10 volume of RnaseA, shaking and suspending for 10-30 s, and water-bathing at 55° C. for 5 min; boiling at 100° C. for 5 min; and shaking and suspending for 10-30 s, centrifuging for 2 min at 12,000 r/min, and taking a supernatant for testing.

Further, the designing primers includes: designing outer primers F3/B3, inner primers FIP/BIP, and a loop primer LB:

F3:
5′-TTGGCTATGGAATTTATCACAT-3′;
B3:
5′-AGCCGCTTTCAAAATCCG-3′;
FIP:
5′-ACCCGAGCCATCCTCTTCTTCTATATTCCTTTTTCTTGGCACATT-3′;
BIP:
5′-TACGACTACGCCTCGCTGTTAGGCTCAATTTTCCCCACT-3′;
and
LB:
5′-GCTTAGGCATGTTTGAGCCAATT-3′.

Further, the establishing a LAMP reaction system includes: establishing the LAMP reaction system based on color determination by using a synthesized pcDNA3.1-Ntsp151 recombinant plasmid as a template, where the reaction system includes: 1×Bst 2.0 DNA polymerase buffer, 6 mmol/L MgSO₄, 0.5 mmol/L dNTP, 0.1 μmol/L F3, 0.1 μmol/L B3, 8 μmol/L FIP, 8 μmol/L BIP, 0.2 μmol/L LB, 8 U Bst 2.0 DNA polymerase, and 2.0 μL template; ddH₂O is added until a total volume of 25 μL; after a reaction is completed, a LAMP amplification product is mixed with a color-producing cap including SYBR Green I; and a color of a reaction solution is observed to obtain a determination result; and
the color-producing cap is prepared by: 10⁴-fold diluting an SYBR Green I dye, adding a dilution to a cap matched with an 8-strip PCR tube, drying the cap at 37° C. for 4 h, and storing the cap in dark at room temperature;
where, a LAMP reaction product is subjected to 1.5% agarose gel electrophoresis (AGE), and a DNA ladder is observed under ultraviolet light.

Further, the optimizing the LAMP reaction system includes: carrying out a LAMP reaction at 60-65° C.

Further, the optimizing the LAMP reaction system includes: carrying out the LAMP reaction at 63° C.

Further, the optimizing the LAMP reaction system includes: carrying out the LAMP reaction with 0-12 mmol/L Mg²⁺.

Further, the optimizing the LAMP reaction system includes: carrying out the LAMP reaction with optimum 6 mmol/L Mg²⁺.

Further, the optimizing the LAMP reaction system includes: carrying out the LAMP reaction for 15-90 min.

Further, the optimizing the LAMP reaction system includes: carrying out the LAMP reaction for 60 min.

The present disclosure has the following beneficial effects:
1) The present disclosure develops a rapid method for extracting tobacco genomic DNA. The whole extraction process can be controlled within 20 min, and is rapid and simple. When used for LAMP test, the effect of this rapid extraction method is not significantly different from that of an ordinary filter cylinder extraction method.
2) The present disclosure establishes the rapid LAMP test technique for tobacco through SYBR Green I, which can complete sample pretreatment and genomic DNA extraction within 20 min, control the LAMP reaction within 45 min, and improve the test sensitivity of Ntsp151 gene to 200 copies per reaction. The test results can be rapidly determined by naked eyes or ultraviolet radiation.
3) The present disclosure designs the LAMP primers according to a conserved region of a screened tobacco-specific Ntsp151 genomic sequence, and establishes the LAMP method based on color determination. The present disclosure can rapidly identify flue-cured tobacco leaves, finished cigarettes, and tea cigarettes, and achieves a detection limit of 200 copies per reaction. The test results of the LAMP method for non-tobacco samples are negative, indicating that the LAMP method has high specificity. Compared with the quantitative polymerase chain reaction (qPCR) method, the LAMP method has a coincidence rate of 96.7%, but the LAMP method only needs about 1 h to obtain the results. Therefore, the LAMP method is rapid and simple, and is of great significance for rapid test of inspection.

The present disclosure is described in further detail below with reference to the drawings and specific implementations.

### BRIEF DESCRIPTION OF THE DRAWINGS

FIGS. 1A-1C are schematic diagrams of an experiment for establishing a LAMP reaction system according to the present disclosure, where PC denotes positive control; NC denotes negative control; and M denotes a DNA ladder;

FIGS. 2A-2C are schematic diagrams of an experiment for determining an optimum concentration of Mg²⁺ for a LAMP system according to the present disclosure, where M denotes a DNA ladder; 0, 2, 4, 6, 8, 10, and 12 denote concentrations of Mg²⁺ added in the LAMP system; and NC denotes negative control;

FIGS. 3A-3C are schematic diagrams of an experiment for determining an optimum reaction temperature for the LAMP system according to the present disclosure, where M denotes the DNA ladder; 60, 61, 62, 63, 64, and 65 denote different reaction temperatures; and NC denotes negative control;

FIGS. 4A-4C are schematic diagrams of an experiment for an effect of a reaction time on LAMP amplification according to the present disclosure, where M denotes the DNA ladder; 15. 30, 45, 60, 75, and 90 denote different reaction times; and NC denotes negative control;

FIGS. 5A-5B are schematic diagrams of an experiment for specificity of the LAMP system according to the present disclosure, where N denotes negative control; P denotes positive control; Nt01 denotes a genomic DNA; and 1 to 17 denote peanut, pea, broad bean, corn, wheat, barley, rice, pepper, eggplant, potato, tomato, petunia, Datura, wolfberry, rape, green tea, and Pu'er tea, respectively;

FIGS. 6A-6D are schematic diagrams of an experiment for sensitivity of the LAMP system according to the present disclosure, where M denotes the DNA ladder; NC denotes negative control; 10⁶, 10⁵, 10⁴, 10³, 10², 10¹, and 10⁰ denote different concentrations of DNA templates; and

FIGS. 7A-7B show LAMP test results of sample extracted by Chelex-100 according to the present disclosure, where N denotes negative control; P denotes positive control; 1. 2, and 3 denote a tobacco genomic DNA extracted by Chelex-100; 4 and 5 denote a tobacco genomic DNA extracted by a kit; FIG. 7A shows naked eye observation results; and FIG. 7B shows ultraviolet radiation results.

### DETAILED DESCRIPTION OF THE EMBODIMENTS

Theoretical explanation: tobacco leaves are rich in a variety of nicotine, protein, pigments, phenols, etc., which often form sticky colloidal substances with DNA during DNA extraction, thus affecting the quality of DNA.

The cetyltrimethylammonium Bromide (CTAB) method and kit extraction method are commonly used for tobacco genome extraction, in addition to the improved polyvinyl pyrrolidone (PVP)-CTAB method. However, these methods are time-consuming, cumbersome, and require professional personnel to operate. Chelex-100 resin is widely used in the field of nucleic acid extraction. It can effectively remove non-nucleic acid organics and chelate high-valent metal ions without affecting the non-metallic ions in the solution. The cell membrane of the cell is lysed under the condition of boiling, and the Chelex-100 particles combined with various organic substances and metal ions are removed by centrifugation. It has been reported that the tobacco genome extracted by Chelex-100 can be used for PCR amplification.

In this study, according to tobacco-specific Ntsp151 gene (ZL 2021 1 0558610.3), namely a transcription factor of tobacco ethylene response factor ERF189, a set of LAMP primers was designed and a visual LAMP amplification system based on SYBR Green I was established. The reaction conditions of the system were optimized, and the sensitivity and specificity of the method were analyzed. Meanwhile, Chelex-100 was combined with proteinase K and RNaseA to establish a rapid extraction method for the tobacco genomic DNA. This method can solve the problem of sticky samples in the extraction process, and the extracted DNA can be directly amplified by LAMP. The rapid tobacco test method features high sensitivity, strong specificity, and is simple and rapid.

### Example

### 1. Materials and Methods

### 1.1 Tobacco and Non-Tobacco Samples

There were 91 tobacco DNA samples tested, including 83 wild tobacco samples and 8 cultivated tobacco samples. There were 17 non-tobacco samples belonging to Solanaceae, Gramineae, Leguminosae, Camelliaceae, and Cruciferae.

TABLE 1
Detail information of the 91 Nicotiana materials

| Sub-genus | Section | Species | PI code | Accessions |
|---|---|---|---|---|
| Rustica | Paniculatae | N. benavedesii | PI 555471 | M001 |
| | | N. cordifolia | PI 555493 | M002 |
| | | N. glauca | PI 555504 | M003 |
| | | N. glauca | PI 307908 | M004 |
| | | N. knightiana | PI 555527 | M005 |
| | | N. paniculata | PI 555545 | M006 |
| | | N. paniculata | PI 555550 | M007 |
| | | N. raimondii | PI 555549 | M008 |
| | | N. solanifolia | PI 555558 | M009 |
| | Rusticae | N. rustica | PI 243561 | M010 |
| Tabacum | Tomentosae | N. glutinosa | PI 555507 | M011 |
| | | N. glutinosa | PI 555505 | M012 |
| | | N. otophora | PI 555542 | M013 |
| | | N. otophora | PI 235553 | M014 |
| | | N. setchellii | PI 555557 | M015 |
| | | N. tomemtosa | PI 574525 | M016 |
| | | N. tomemtosiformis | PI 555572 | M017 |
| Petunioides | Undulatae | N. undulata | PI 555574 | M018 |
| | | N. undulata | PI 555575 | M019 |
| | | N. wigandioides | PI 302471 | M020 |
| | Alatae | N. alata | PI 42334 | M021 |
| | | N. bonariensis | PI 555489 | M022 |
| | | N. forgetiana | PI 555501 | M023 |
| | | N. langsdorffii | PI 42337 | M024 |
| | | N. langsdorffii | PI 555529 | M025 |
| | | N. longiflora | PI 555531 | M026 |
| | | N. longiflora | PI 555532 | M027 |
| | | N. longiflora | PI 555533 | M028 |
| | | N. plumbaginifolia | PI 555548 | M029 |
| | | N. plumbaginifolia | PI 302476 | M030 |
| | | N. plumbaginifolia | PI 302478 | M031 |
| | | N. sylvestris | PI 555569 | M032 |
| | | N. sylvestris | PI 555570 | M033 |
| | | N. sylvestris | PI 555571 | M034 |
| | Nudicaulisae | N. nudicaulis | PI 555540 | M035 |
| | Repandae | N. repanda | PI 555552 | M036 |
| | | N. repanda | PI 555551 | M037 |
| | | N. stocktonii | PI 555538 | M038 |
| | | N. stocktonii | PI 555539 | M039 |
| | | N. stocktonii | PI 555560 | M040 |
| | Noctiflorae | N. noctiflora | PI 417918 | M041 |
| | | N. noctiflora | PI 475832 | M042 |
| | | N. petunioides | PI 555547 | M043 |
| | Acuminatae | N. acuminata | PI 555477 | M044 |
| | | N. attenuata | PI 555476 | M045 |
| | | N. corymbosa | PI 114824 | M046 |
| | | N. pauciflora | PI 555546 | M047 |
| | Bigelovianae | N. bigelovii | PI 555485 | M048 |
| | | N. clavelandii | PI 555491 | M049 |
| | Suaveolensae | N. africana | PI 555472 | M050 |
| | | N. amplxicaulis | PI 271989 | M051 |
| | | N. amplxicaulis | PI 555682 | M052 |
| | | N. benthamiana | PI 555478 | M053 |
| | | N. cavicola | PI 271990 | M054 |
| | | N. debneyi | PI 503320 | M055 |
| | | N. excelsior | PI 224063 | M056 |
| | | N. excelsior | PI 555685 | M057 |
| | | N. goodspeedii | PI 241012 | M058 |
| | | N. gossei | PI 230953 | M059 |
| | | N. linearis | PI 555530 | M060 |
| | | N. miersii | PI 555537 | M061 |
| | | N. maritima | PI 555535 | M062 |
| | | N. megalosiphon | PI 555536 | M063 |
| | | N. megalosiphon | PI 555688 | M064 |
| | | N. occidentalis | PI 271991 | M065 |
| | | N. occidentalis | PI 555687 | M066 |
| | | N. occidentalis | PI 555541 | M067 |
| | | N. occidentalis | PI 555690 | M068 |
| | | N. rosulata | PI 244635 | M069 |
| | | N. rosulata | PI 244624 | M070 |
| | | N. rosulata | PI 244628 | M071 |
| | | N. rotundifolia | PI 555553 | M072 |
| | | N. rotundifolia | PI 555691 | M073 |
| | | N. suaveolens | PI 555500 | M074 |
| | | N. suaveolens | PI 555565 | M075 |
| | | N. suaveolens | PI 230960 | M076 |
| | | N. umbratica | PI 271993 | M077 |
| | | N. velutina | PI 244638 | M078 |
| | | N. velutina | PI 244630 | M079 |
| | | N. velutina | PI 244631 | M080 |
| | | N. acuminata | PI 555469 | M081 |
| | | N. acuminata | PI 42347 | M082 |
| | | N. benthamiana | PI 555684 | M083 |

| Names | Types | Accessions |
|---|---|---|
| K326 | Flue-cured | M084 |
| HD | Flue-cured | M085 |
| TN90 | Burley | M086 |
| Burley 21 | Burley | M087 |
| Basma Xanthi | Oriental | M088 |
| Sumsun NN | Oriental | M089 |
| Beinhart1000-1 | Cigar | M090 |
| Florida301 | Cigar | M091 |

Note: The 83 wild tobacco species were counted by PI code, and there were wild tobacco species with the same name but different PI codes.

### 1.2 Main Reagents and Instruments

The genomic DNA extraction kit was purchased from Tiangen Biotechnology Co., Ltd. Chelex-100 was purchased from Bio-Rad. Proteinase K was purchased from ThermoFisher. RnaseA was purchased from ThermoFisher. Bst 2.0 polymerase was purchased from NEB. SYBR Green I dye was purchased from Sigma. dNTPs (10 μmol/L) was purchased from Takara. StepOne real-time PCR instrument was purchased from ABI. The gel imaging system was purchased from Beijing Liuyi Biotechnology Co., Ltd. The fluorescence tester was purchased from Hangzhou Allsheng Instrument Co., Ltd. DK-8D thermostatic bath was purchased from Hangzhou Bioer Technology Co., Ltd. The recombinant plasmid positive reference pcDNA3.1-Ntsp151 and primers were synthesized by General Biology (Anhui) Co., Ltd.

### 1.3 Extraction Method of Tobacco Genomic DNA

10-15 mg of a tobacco sample was weighed and added into an Eppendorf (EP) tube, 5% of a Chelex-100 suspension was added, grinding was carried out with a grinding rod for 1-2 min, and shaking and suspending was carried out for 10-30 s. 1/10 volume of proteinase K and 1/10 volume of RnaseA were added, shaking and suspending was carried out for 10-30 s, and water-bathing was carried out at 55ºC for 5 min. Boiling was carried out at 100° C. for 5 min. Shaking and suspending was carried out for 10-30 s, centrifuging was carried out for 2 min at 12,000 r/min, and a supernatant was take for test.

1.4 Design of LAMP and qPCR Primers for Tobacco Ntsp151 Gene

According to the previously discovered tobacco-specific Ntsp151 gene sequence, a set of specific amplification test primers was screened through the online software Primer Explore V5 (http://primerexplorer.jp/lampv5e/index.html) based on the LAMP primer design principle, including outer primers F3/B3, inner primers FIP/BIP, and loop primer LB, as shown in Table 2. According to the Ntsp151 gene sequence, a set of fluorescence quantitative PCR primers tested by SYBR Green I fluorescence quantitative method was designed using Oligo7, as shown in Table 2. The freeze-dried powder of the synthesized primer was centrifuged instantaneously, and a proper amount of deionized water was added to dissolve the DNA to obtain 100 μmol/L of a stock solution. F3, B3, LB, Ntsp151-qF1, and Ntsp151-qR1 were diluted by 1:10 to obtain 10 μmol/L of a working solution. The working solution was stored at −20° C. for standby.

TABLE 2
Primer sequences of LAMP and qPCR

| | Primer | Nucleotide sequence (5′-3′) |
|---|---|---|
| 1 | F3 | TTGGCTATGGAATTTATCACAT |
| 2 | B3 | AGCCGCTTTCAAAATCCG |
| 3 | FIP(F1c + F2) | ACCCGAGCCATCCTCTTCTTCTATATTCCTTTTTCTTGGCACATT |
| 4 | BIP(B1c + B2) | TACGACTACGCCTCGCTGTTAGGCTCAATTTTCCCCACT |
| 5 | LoopB(LB) | GCTTAGGCATGTTTGAGCCAATT |
| 6 | Ntsp151-qF1 | CTCGCTGTTACTCTAGCC |
| 7 | Ntsp151-qR1 | ACTTACGAGACCGCAGA |

### 1.5 Establishment and Optimization of LAMP Reaction System

The LAMP reaction system was established based on color determination by using a synthesized pcDNA3.1-Ntsp151 recombinant plasmid as a template. The reaction system was formed by 1×Bst 2.0 DNA polymerase buffer, 6 mmol/L MgSO₄, 0.5 mmol/L dNTP, 0.1 μmol/L F3, 0.1 μmol/L B3, 8 μmol/L FIP, 8 μmol/L BIP, 0.2 μmol/L LB, 8 U Bst 2.0 DNA polymerase, and 2.0 μL template. ddH₂O was added until a total volume of 25 μL. After a reaction was completed, a LAMP amplification product was mixed with a color-producing cap including SYBR GreenI. A color of a reaction solution was observed to obtain a determination result.

The color-producing cap was prepared by 10⁴-fold diluting an SYBR Green I dye, adding a dilution to a cap matched with an 8-strip PCR tube, drying the cap at 37° C. for 4 h, and storing the cap in dark at room temperature.

A LAMP reaction product is subjected to 1.5% agarose gel electrophoresis (AGE), and a DNA ladder is observed under ultraviolet light.

A LAMP reaction was carried out at 60-65° C., and the color of a reaction product and a DNA ladder were observed to determine the optimum reaction temperature. At the optimum reaction temperature, the amplification products 15, 30, 45, 60, 75, and 90 min were collected at to determine the optimum reaction time of the system. Mg²⁺ of different concentrations (0, 2, 4, 6, 8, 10, 12 mmol/L) were added into the LAMP system, and the optimum concentration of Mg²⁺ was determined by observing the color of the product and DNA electrophoresis results.

### 1.6 LAMP Specificity Test Primers

The optimized LAMP method was used to test the genomic DNAs of the 17 non-tobacco crops, including peanut, pea, broad bean, corn, wheat, barley, rice, pepper, eggplant, potato, tomato, petunia, Datura, wolfberry, rape, green tea, and Pu'er tea. The colors of the amplification products were observed by naked eyes, and the positive and negative results were determined so as to evaluate the specificity of the system.

### 1.7 Determination of LAMP Test Sensitivity

The copy number of the plasmid was calculated using the constructed pcDNA3.1-Ntsp151 recombinant plasmid as a template. The plasmid was gradiently diluted by 10⁶, 10⁵, 10⁴, 10³, 10², 10¹, 10⁰ copies/μL. It was added to the LAMP system as a template, 2.0 μL for each reaction hole. The test results were observed with naked eyes, and the positive and negative results were determined, so as to evaluate the sensitivity.

1.8 Comparison of LAMP and qPCR Test Methods

The 91 tobacco samples of different species and genera were tested by LAMP and qPCR so as to verify the coincidence rate of the two methods for tobacco test.

### 2. Results

### 2.1 Establishment and Optimization of LAMP Reaction System

### 2.1.1 Establishment of LAMP Reaction System

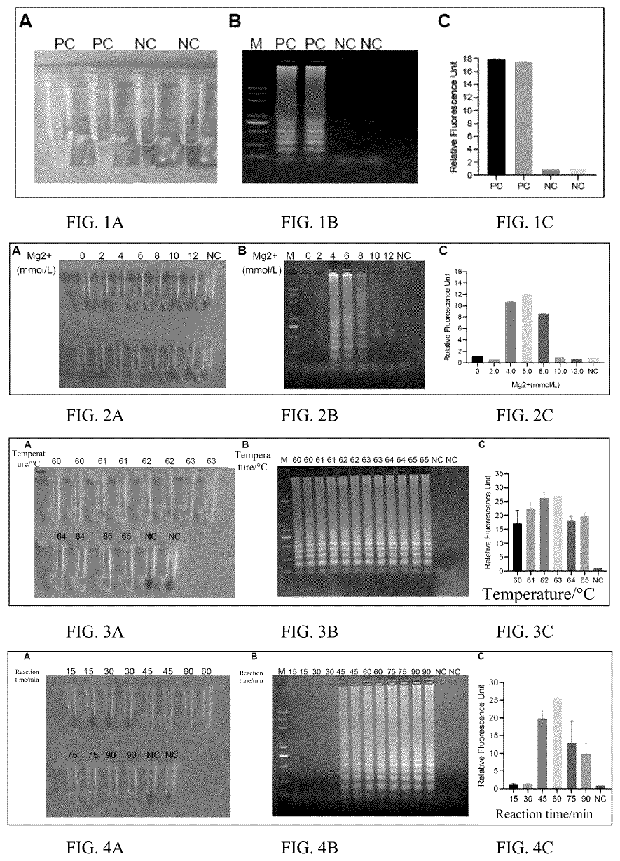

The LAMP amplification primers for the Ntsp151 gene were designed, and the positive reference for gene amplification was constructed, so as to preliminarily verify the amplification reaction of the primers, as shown in FIGS. 1A-1C. It can be seen from the figure that compared with the control group, the experimental group added with the positive reference had a large number of double-stranded DNAs, which emitted obvious green fluorescence after combined with SYBR Green I. The control group did not include double-stranded DNAs, and the color of the reaction solution was orange, which was obviously different from the color of the experimental group. Meanwhile, the DNA ladder and fluorescence test results of the LAMP amplification products were consistent with the color-producing results. The fluorescence values of the products in the experimental group and the control group were tested. The fluorescence value of the experimental group was 17.8 times that of the control group.

### 2.1.2 Optimization of Mg²⁺ Concentration

In order to study the effect of different concentrations of Mg²⁺ on the LAMP reaction system, 7 concentration gradients, namely 0, 2, 4, 6, 8, 10, and 12 mmol/L were selected. The determination results were obtained by comparing the colors of the amplification products, the DNA ladders formed by electrophoresis analysis and the fluorescence values of the end-point test product, as shown in FIGS. 2A-2C. It can be seen from the figure that when the concentrations of the added Mg²⁺ were 4, 6 and 8 mmol/L, there was an obvious amplification reaction. When the concentration of Mg²⁺ was too low (<4 mmol/L), the amplification reaction was not effectively initiated. When the concentration of Mg²⁺ was too high, the amplification reaction was inhibited. Based on the results of color producing, electrophoresis and fluorescence value, the optimum concentration of Mg²⁺ in the LAMP reaction system was confirmed to be 6 mmol/L.

### 2.1.3 Determination of Optimum Reaction Temperature

In order to study the effect of different temperatures on the LAMP reaction system, 6 temperatures, namely 60° C., 61° C., 62° C., 63° C., 64° C., and 65° C., were selected for amplification, and the results are shown in FIGS. 3A-3C. It can be seen from the figure that when the amplification was carried out at different temperatures, the color results and electrophoresis results of the products had no obvious differences. However, the fluorescence test results showed that the fluorescence intensity of amplification products at 63ºC was stronger than that of other groups, indicating that there were more double-stranded DNAs at this time. Therefore, 63° C. was selected as the optimum reaction temperature for the primer.

### 2.1.4 Determination of LAMP Reaction Time

In order to determine the optimum time for LAMP amplification, 6 time points, namely 15 min, 30 min, 45 min, 60 min, 75 min, and 90 min, were selected. The amplification level of the target DNA was determined by product color, electrophoresis test, and fluorescence test, and the results are shown in FIGS. 4A-4C. It can be seen from the figure that the 15 min and 30 min amplifications showed no obvious target DNA ladder and no change in the product color, and the fluorescence test result was consistent with those of the above two time points. At 45 min, there was obvious product amplification. With the extension of time, at 60 min, the product amplification was still obvious. At 75 min and 90 min, the target product amplification was not obvious. Therefore, 60 min was selected as the optimum amplification time of the system.

### 2.2 LAMP Specificity Test

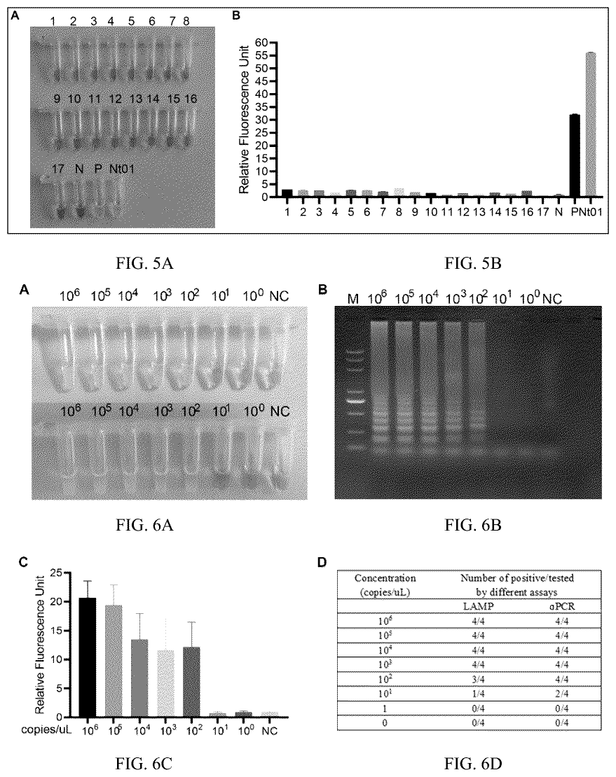

The established LAMP method was used to test the 17 non-tobacco samples and 1 tobacco sample. The fluorescence color and fluorescence value of the product were observed, and the results are shown in FIGS. 5A-5B. The colors of the amplified products of the positive control and tobacco sample of the system were green, and the results of the negative reference and 17 non-tobacco samples were negative. The test results of the fluorescence value were consistent with the color producing results, indicating that the LAMP test method has high specificity.

### 2.3 LAMP Sensitivity Test

The prepared positive reference pcDNA3.1-Ntsp151 recombinant vector was diluted gradiently, and 2.0 μL template was added to each reaction system. The amplification results are shown in FIGS. 6A-6D. The detection limit of the LAMP reaction was 200 copies per reaction.

### 2.4 Test of Different Types of Tobacco Samples

A total of 91 tobacco samples were tested by the established LAMP method, including 83 wild tobacco belonging to three subgenera, namely rustica, Tabacum, and petunioides, and 8 cultivated tobacco. The results are shown in Table 3. The positive rate of the LAMP method was 96.7% (88/91), and the positive rate of the qPCR method was 100% (91/91). The coincidence rate of the LAMP method was 96.7%. The reason for the undetected samples might be that the sample concentration was too low, or there were genome mutations in these samples.

TABLE 3
Various types of tobacco samples for LAMP test

| | | LAMP | | qPCR | |
|---|---|---|---|---|---|
| Samples | n | Positive | Negative | Positive | Negative |
| Rustica | 10 | 10 | 0 | 10 | 0 |
| Tabacum | 7 | 7 | 0 | 7 | 0 |
| Petunioides | 66 | 63 | 3 | 66 | 0 |
| Cultivated tobacco | 8 | 8 | 0 | 8 | 0 |

### 2.5 Extraction of Tobacco Genomic DNA

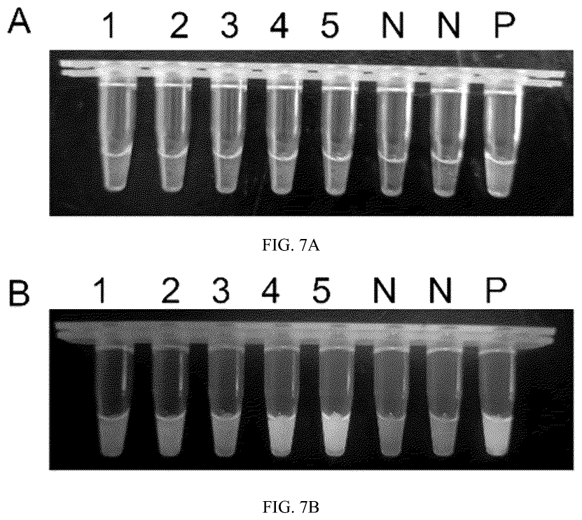

The genomes in tobacco samples were extracted by the kit extraction method and the Chelex-100 method, respectively. The extracted genomic DNAs were added into the established LAMP reaction solution, and the color-producing results of the LAMP system were observed, as shown in FIGS. 7A-7B. It can be seen from the figure that there was no obvious difference between the two extraction methods.

The above described is only part of the specific embodiments of the present disclosure. (Since the present disclosure involves a numerical range, the embodiments cannot be exhaustive, and the scope of protection recorded in the present disclosure includes the numerical range and other technical points of the present disclosure). The specific contents or common sense known in the solution are not described herein (including but not limited to simplified forms, abbreviations, and units commonly used in the field). Those skilled in the art should understand that the above embodiments are not intended to limit the present disclosure in any form, and that any technical solutions obtained by means of equivalent replacement or equivalent transformation should fall within the protection scope of the present disclosure. The scope of protection claimed in this application shall be subject to the content of the claims, and the specific implementations in the description may be intended to interpret the content of the claims.

What is claimed is:

1. A visual loop-mediated isothermal amplification (LAMP) method for a rapid test of a tobacco, comprising: extracting a genomic DNA of the tobacco, designing primers, establishing a LAMP reaction system, and optimizing the LAMP reaction system.

2. The visual LAMP method for the rapid test of the tobacco according to claim 1, wherein the extracting the genomic DNA of the tobacco comprises: weighing and adding 10-15 mg of a tobacco sample into an Eppendorf (EP) tube, adding 5% of a Chelex-100 suspension, grinding with a grinding rod for 1-2 min, and shaking and suspending for 10-30 s; adding 1/10 volume of a proteinase K and 1/10 volume of a RnaseA, shaking and suspending for 10-30 s, and water-bathing at 55ºC for 5 min; boiling at 100° C. for 5 min; and shaking and suspending for 10-30 s, centrifuging for 2 min at 12,000 r/min, and taking a supernatant for testing.

3. The visual LAMP method for the rapid test of the tobacco according to claim 1, wherein the designing the primers comprises: designing outer primers F3/B3, inner primers FIP/BIP, and a loop primer LB:
F3:
5′-TTGGCTATGGAATTTATCACAT-3′, as shown in SEQ ID NO: 1;
B3:
5′-AGCCGCTTTCAAAATCCG-3′, as shown in SEQ ID NO: 2;
FIP:
5′-ACCCGAGCCATCCTCTTCTTCTATATTCCTTTTTCTTGGCACATT-3′, as shown
in SEQ ID NO: 3;
BIP:
5′-TACGACTACGCCTCGCTGTTAGGCTCAATTTTCCCCACT-3′, as shown in SEQ
ID NO: 4;
and
LB:
5′-GCTTAGGCATGTTTGAGCCAATT-3′, as shown in SEQ ID NO: 5.

4. The visual LAMP method for the rapid test of the tobacco according to claim 1, wherein the establishing the LAMP reaction system comprises: establishing the LAMP reaction system based on a color determination by using a synthesized pcDNA3.1-Ntsp151 recombinant plasmid as a template, wherein the LAMP reaction system comprises: 1×Bst 2.0 DNA polymerase buffer, 6 mmol/L MgSO₄, 0.5 mmol/L dNTP, 0.1 μmol/L F3, 0.1 μmol/L B3, 8 μmol/L FIP, 8 μmol/L BIP, 0.2 μmol/L LB, 8 U Bst 2.0 DNA polymerase, and 2.0 μL of the template; ddH₂O is added until a total volume of 25 μL; after a reaction is completed, a LAMP amplification product is mixed with a color-producing cap comprising an SYBR GreenI; and a color of a reaction solution is observed to obtain a determination result; and
the color-producing cap is prepared by: 10⁴-fold diluting an SYBR Green I dye, adding a dilution to a cap matched with an 8-strip polymerase chain reaction (PCR) tube, drying the cap matched with the 8-strip PCR tube at 37° C. for 4 h, and storing the cap matched with the 8-strip PCR tube in dark at a room temperature;
wherein, a LAMP reaction product is subjected to a 1.5% agarose gel electrophoresis (AGE), and a DNA ladder is observed under an ultraviolet light.

5. The visual LAMP method for the rapid test of the tobacco according to claim 1, wherein the optimizing the LAMP reaction system comprises: carrying out a LAMP reaction at 60-65° C.

6. The visual LAMP method for the rapid test of the tobacco according to claim 5, wherein the optimizing the LAMP reaction system comprises: carrying out the LAMP reaction at 63° C.

7. The visual LAMP method for the rapid test of the tobacco according to claim 1, wherein the optimizing the LAMP reaction system comprises: carrying out a LAMP reaction with 0-12 mmol/L Mg²⁺.

8. The visual LAMP method for the rapid test of the tobacco according to claim 7, wherein the optimizing the LAMP reaction system comprises: carrying out the LAMP reaction with 6 mmol/L Mg²⁺.

9. The visual LAMP method for the rapid test of the tobacco according to claim 1, wherein the optimizing the LAMP reaction system comprises: carrying out a LAMP reaction for 15-90 min.

10. The visual LAMP method for the rapid test of the tobacco according to claim 9, wherein the optimizing the LAMP reaction system comprises: carrying out the LAMP reaction for 60 min.


SEQUENCE LISTING (7 sequences)

SEQ ID NO: 1 - DNA, 22 bp, synthetic construct
  source 1..22: mol_type = other DNA
ttggctatggaatttatcacat

SEQ ID NO: 2 - DNA, 18 bp, synthetic construct
  source 1..18: mol_type = other DNA
agccgctttcaaaatccg

SEQ ID NO: 3 - DNA, 45 bp, synthetic construct
  source 1..45: mol_type = other DNA
acccgagccatcctcttcttctatattcctttttcttggcacatt

SEQ ID NO: 4 - DNA, 39 bp, synthetic construct
  source 1..39: mol_type = other DNA
tacgactacgcctcgctgttaggctcaattttccccact

SEQ ID NO: 5 - DNA, 23 bp, synthetic construct
  source 1..23: mol_type = other DNA
gcttaggcatgtttgagccaatt

SEQ ID NO: 6 - DNA, 18 bp, synthetic construct
  source 1..18: mol_type = other DNA
ctcgctgttactctagcc

SEQ ID NO: 7 - DNA, 17 bp, synthetic construct
  source 1..17: mol_type = other DNA
acttacgagaccgcaga